(19) United States
(12) Patent Application Publication
(10) Pub. No.: US 2025/0145925 A1
(43) Pub. Date: May 8, 2025

(54) FLEXIBLE BIOPROCESSING VESSEL AND RIGID SUPPORT STRUCTURE

(71) Applicant: Pall Technology UK Limited, Portsmouth (GB)

(72) Inventors: Bart Heeren, Brussel (BE); Jorrit Sevenants, Hoegaarden (BE); Steven van den Berghe, Hoegaarden (BE); Koen Eraly, Hoegaarden (BE); Arnaud De Boulard, Hoegaarden (BE)

(73) Assignee: Pall Technology UK Limited, Portsmouth (GB)

(21) Appl. No.: 18/388,028

(22) Filed: Nov. 8, 2023

(51) Int. Cl. C12M 1/00; C12M 1/06; C12M 3/00

(52) U.S. Cl. CPC C12M 23/26; C12M 23/46; C12M 27/02; C12M 29/14

(57) ABSTRACT

An apparatus for supporting a flexible bioprocessing vessel includes a rigid body having a hexagonal interior that includes a bottom surface and six sides surrounding the bottom surface, the hexagonal interior having a substantially open top. The apparatus further includes a selectively openable door allowing access to the hexagonal interior and a stand portion attached to the rigid body, the stand portion allowing access to an underside of the rigid body. The hexagonal interior is configured to receive a hexagonal flexible bioprocessing vessel having a liquid level height to width ratio of ≤1.

### CROSS REFERENCE TO RELATED APPLICATIONS

The present patent application is related to concurrently filed, copending, and commonly assigned U.S. patent application Ser. No. ______, entitled “APPARATUS, SYSTEM, AND METHOD FOR TRANSPORTING AND INSTALLING A FLEXIBLE BIOPROCESSING VESSEL” (Attorney Docket No.: P2023-3885-US01), the disclosure of which is incorporated herein by reference.

### BACKGROUND

### Technical Field

Embodiments of the invention relate generally to bioprocessing, and, more particularly, to a flexible bioprocessing vessel and a rigid support structure for a flexible bioprocessing vessel.

### Discussion of Art

Mixers and bioreactors are often employed to carry out biochemical and biological processes and/or manipulate liquids and other products of such processes. These devices typically utilize single-use vessels e.g., flexible or collapsible bags that are supported by an outer rigid structure such as a stainless-steel housing/tank. As will be appreciated, use of sterilized single use bags eliminates the time-consuming step of cleaning the tank after each use and reduces the chance of contamination.

In use, a disposable/single-use bag is positioned within the rigid tank and filled with the desired fluid for processing. An impeller assembly that includes a rotating impeller having one or more blades is disposed within the bag and is used to mix the fluid. Existing impeller systems are either top-driven, having a shaft that extends downwardly into the bag, on which one or more impellers are mounted, or bottom-driven, having an impeller disposed in the bottom of the bag that is driven by, for example, a magnetic drive system positioned outside the bag.

Support structures/tanks may be relatively large, having capacities of 2000 L-3000 L or more. As will be appreciated, such tanks are often installed in existing cleanroom spaces. To that end, tanks should be compatible with existing cleanroom architecture, i.e., installation should not require expensive and time-consuming interior modifications. For example, a 3000 L tank should be able to pass through a double door having a height of 213.4 cm (7 ft) and a width of 182.9 cm (6 ft). Many cylindrical and cuboid/cubical tanks having a capacity greater than 2000 L, however, have a height that makes such passage impossible.

Moreover, tank height is dictated in large part by the size of the flexible single-use bags that are supported within the tank. Known 2000 L and 3000 L bags have undesirable height to width ratios greater than 1, i.e., they are taller than they are wide. For example, 2000 L cubical mixer bags have a height to width ratio of 1.6. Known 3000 L cubical mixer bags have a height to width ratio of 2.4. The width of such bags cannot be easily increased to reduce height as the width of available flexible film is limited.

These flexible single-use bags must also be shipped and installed in the tank. 2000 L and 3000 L flexible bags are relatively large and given their size and flexible nature, may be challenging to install. Known tanks do not include features that facilitate alignment of a flexible vessel to provide an ease of installation.

Mixing efficiency is also important in vessel and tank design. Overall mixing efficiency is depending on mixing power and the capability to dissipate mixing power across a volume of fluid in the vessel. For example, relatively taller than wide mixers, with a bottom or top mounted impeller, typically are very limited in their capability to dissipate mixing powder in the top or bottom section of the vessel respectively. Further, cuboid/cubical vessels typically do not result in vortex formation but generate high levels of turbulence. In contrast, cylindrical vessels generate a vortex but low levels of turbulence. As a result, cuboid and cubical vessels/tanks are generally suitable for use with floating powders (media) and cylindrical vessels/tanks for use with sinking powders (buffer). As such, these vessels lack ideal mixing versatility.

In view of the above, there is a need for a flexible bioprocessing vessel and rigid support structure that allows for installation in existing cleanrooms without having to make interior modifications. There is also a need for a flexible bioprocessing vessel and rigid support structure that allow for versatile and efficient mixing in a variety of applications, and that provide an ease of vessel installation within the rigid support structure.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, an apparatus for supporting a flexible bioprocessing vessel includes a rigid body having a hexagonal interior that includes a bottom surface and six sides surrounding the bottom surface, the hexagonal interior having a substantially open top. The apparatus further includes a selectively openable door allowing access to the hexagonal interior and a stand portion attached to the rigid body, the stand portion allowing access to an underside of the rigid body. The hexagonal interior is configured to receive a hexagonal flexible bioprocessing vessel having a liquid level height to width ratio of 1.

In an embodiment, the hexagonal interior is configured to receive a 2000 L hexagonal flexible bioprocessing vessel and the apparatus has an overall width of about 170 cm and an overall height of about 202 cm.

In another embodiment, the hexagonal interior is configured to receive a 3000 L hexagonal flexible bioprocessing vessel and the apparatus has an overall width of about 195 cm and an overall height of about 210 cm.

In another embodiment, the bottom surface has at least one alignment aperture configured to engage at least one locator tab on a base portion of the hexagonal flexible bioprocessing vessel to facilitate alignment of the hexagonal flexible bioprocessing vessel within the hexagonal interior.

In yet another embodiment, the rigid body has a hexagonal exterior formed by six rigid panels, the six rigid panels also defining the six sides of the hexagonal interior.

In another embodiment, one of the six rigid panels faces a front of the apparatus and includes the selectively openable door.

In another embodiment, the apparatus includes an opening in the rigid body located below the selectively openable door, the opening facilitating connection of one or more fluid lines and/or probes to the hexagonal flexible bioprocessing vessel.

In another embodiment, rigid body may include a sill located between the selectively openable door and the bottom surface of the hexagonal interior, the sill having a notch configured to receive the hexagonal flexible bioprocessing vessel in a folded or collapsed state to facilitate installation of the hexagonal flexible bioprocessing vessel on the bottom surface of the hexagonal interior.

In another embodiment, the bottom surface includes at least one opening configured to allow tubing from the hexagonal flexible bioprocessing vessel to exit the hexagonal interior.

According to an aspect of the invention, a hexagonal flexible bioprocessing vessel includes six flexible panels forming sides of the hexagonal flexible bioprocessing vessel and a top panel adjoining the six flexible panels, the top panel forming a top of the hexagonal flexible bioprocessing vessel. The vessel further includes a bottom panel adjoining the six flexible panels, the bottom panel located on an opposite end of the hexagonal flexible bioprocessing vessel from the top surface, and forming a bottom of the hexagonal flexible bioprocessing vessel, the six flexible panels, top surface, and bottom panel defining an interior cavity configured for processing a fluid and at least one fluid input and at least one fluid output for adding and removing fluid to and from the interior cavity of the hexagonal flexible bioprocessing vessel respectively. The hexagonal flexible bioprocessing vessel has a liquid level height to width ratio of 1.

In an embodiment, the hexagonal flexible bioprocessing vessel has a 2000 L capacity and a liquid level height to width ratio of about 0.91.

In another embodiment, the hexagonal flexible bioprocessing vessel has a width of about 150 cm, an overall height of about 151 cm, and a liquid level height of about 137 cm.

In an embodiment, the hexagonal flexible bioprocessing vessel has a 3000 L capacity and a liquid level height to width ratio of about 0.86.

In an embodiment, the hexagonal flexible bioprocessing vessel has a width of about 175 cm, an overall height of about 159 cm, and a liquid level height of about 151 cm.

In an embodiment, the bottom panel and/or the top panel may be a separate panel.

In an embodiment, each of the six flexible panels is thermally welded to adjacent panels.

In an embodiment, the interior cavity includes an impeller and the top panel includes a selectively removable impeller cover to protect the impeller during transportation of the hexagonal flexible bioprocessing vessel.

In an embodiment, the hexagonal flexible bioprocessing vessel may be folded for storage and/or transportation and unfolded for installation on a bottom surface of a rigid support structure.

### BRIEF DESCRIPTION OF THE FIGURES

The present invention will be better understood from reading the following description of non-limiting embodiments, with reference to the attached drawings, wherein below:

FIG. 1 is a perspective view of a rigid support structure according to an embodiment of the present invention;

FIG. 2 is a top view of the rigid support structure of FIG. 1 depicting a hexagonal interior having a bottom surface with alignment apertures for aligning a flexible vessel placed within the hexagonal interior;

FIG. 3 is a front view of the rigid support structure of FIG. 1;

FIG. 4 is a side view of the rigid support structure of FIG. 1;

FIG. 5 is a front view of the rigid support structure of FIG. 1 without its selectively removable door;

FIG. 6 is an exploded perspective of a flexible bioprocessing vessel according to an embodiment of the invention illustrating individual panels for assembly;

FIG. 7 is a perspective view of an assembled flexible bioprocessing vessel according to an embodiment of the invention;

FIG. 8 is another perspective view of a flexible bioprocessing vessel depicting the vessel secured to an apparatus for transporting and installing the vessel in a rigid support structure according to an embodiment of the invention;

FIG. 9 is a perspective view of the apparatus for transporting and installing the vessel in a rigid support structure of FIG. 8;

FIG. 10 is an illustration of users transporting and installing a flexible bioprocessing vessel into a rigid support structure according to embodiments of the invention;

FIG. 11 is another illustration of the installation of a flexible bioprocessing vessel into a rigid support structure, depicting a tab engaging an alignment aperture in the rigid support structure to facilitate alignment of the vessel, according to embodiments of the invention;

FIG. 12 is yet another illustration of the installation of a flexible bioprocessing vessel into a rigid support structure according to embodiments of the invention.

FIG. 13 is an illustration of the installation of a flexible bioprocessing vessel into a rigid support structure, depicting a foot portion engaging a slot in the rigid support structure to facilitate alignment of the vessel, according to embodiments of the invention;

FIG. 14 is another illustration of the installation of a flexible bioprocessing vessel into a rigid support structure according to the embodiment of FIG. 13;

FIG. 15 is yet another illustration of the installation of a flexible bioprocessing vessel into a rigid support structure according to the embodiment of FIG. 13.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass vessels, (e.g., bioprocessing vessels), having a wall or a portion of a wall that is flexible, single-use flexible bags, as well as other containers or conduits commonly used in biological or biochemical processing, including, for example, cell culture/purification systems, fermentation systems, mixing systems, media/buffer preparation systems, and filtration/purification systems.

As used herein, the term “bag” means a flexible or semi-rigid vessel used, for example, as a mixer or bioreactor for the contents within.

Embodiments may be utilized in connection with a wide variety of biological and chemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device. Certain embodiments may be suitable for use in other industries/applications where size, ease of installation, and/or efficient, versatile mixing of fluids is desirable.

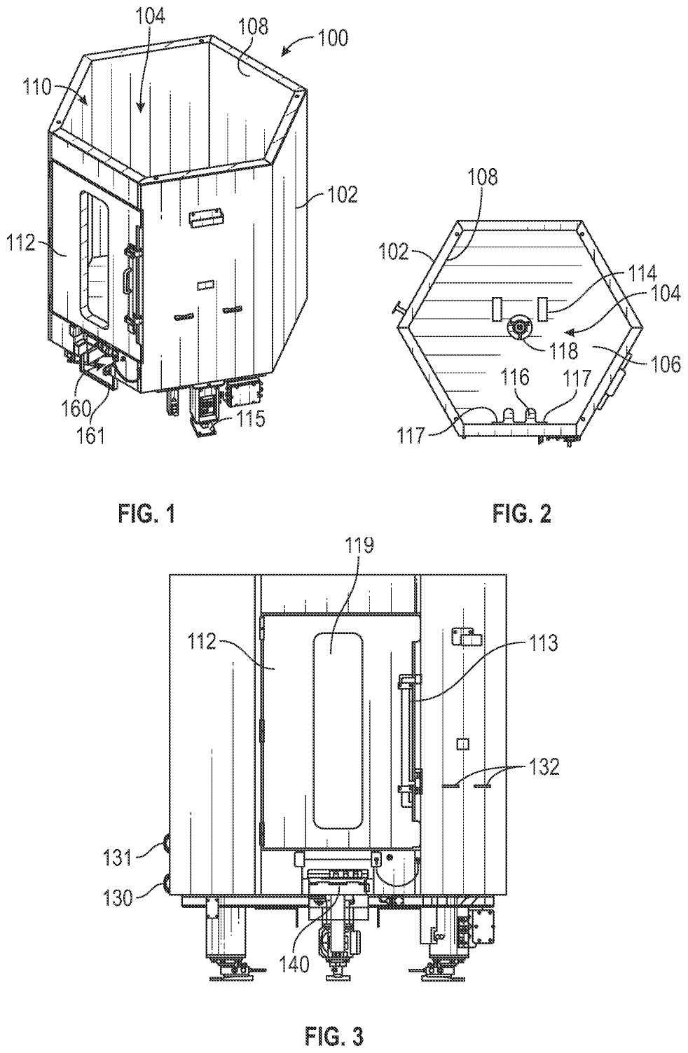

Referring now to FIG. 1, an apparatus 100 for supporting a flexible bioprocessing vessel according to an embodiment of the invention is depicted. The apparatus 100 includes a rigid body 102 having a hexagonal interior 104 that includes a bottom surface 106 and six rigid panels 108 that form sides surrounding the bottom surface 106. The hexagonal interior 104 has a substantially open top 110 and a selectively openable door 112 which includes a polycarbonate viewing window 119. Of course, in embodiments, the viewing window 119 may be of varying size, shape and material without departing from the invention. As will be appreciated, the selectively openable door 112 allows access to the hexagonal interior 104, which is configured to receive a hexagonal flexible bioprocessing vessel 300 (FIG. 7).

In the depicted embodiment, the rigid body 102 has a hexagonal exterior formed by six rigid panels 108, the six rigid panels 108 also define the six sides of the hexagonal interior 104. As will be appreciated, however, in other embodiments the exterior of the rigid body 102 may have a shape, structure, or configuration that departs from the hexagonal interior 104. For example, in embodiments, the exterior may be a separate structure from the hexagonal interior, e.g., the rigid body may have a square, cuboid, cylindrical, or other shaped exterior while maintaining its hexagonal interior 104. In embodiments, the selectively openable door 112 is located on one of the six rigid panels that faces a front of the apparatus.

Referring again to FIG. 1, the apparatus 100 also includes a space or opening 160 in the rigid body 102 located below the selectively openable door 112, the opening 160 facilitates connection of tubing, e.g., one or more fluid lines and/or probes to a hexagonal flexible bioprocessing vessel. That is, it allows tubing to exit the hexagonal interior. In a specific embodiment, the opening 160 allows access to a sample line port 309, sensor ports 313, and the drain port 306 (FIG. 7).

The rigid body 102 further includes a stand portion 115 attached to the rigid body 102 which allows for access to the space below the body. In the depicted embodiment, the stand portion 115 includes a plurality of legs. The number of legs may vary, however, and certain embodiments may utilize a structure other than legs to raise the rigid body 102 or otherwise allow access to an underside of the rigid body. In certain embodiments, the legs are height adjustable and may include load cells. As will be appreciated, access to the space below the rigid body 102 is important in that the mixing motor, seat locking mechanism, and forklift channels (among other features) are located beneath the rigid body 102.

As mentioned, the apparatus 100 is configured to receive a hexagonal flexible bioprocessing vessel. In particular, a hexagonal flexible bioprocessing vessel having total bag height and a liquid level (e.g., maximum liquid level) height to width ratio of ≤1. In embodiments, the hexagonal interior 104 is configured to receive a 2000 L hexagonal flexible bioprocessing vessel, and the apparatus has an overall width of about 172 cm, a depth of about 165 cm (including the probe support), and an overall height of about 203 cm. In another embodiment, the hexagonal interior 104 is configured to receive a 3000 L hexagonal flexible bioprocessing vessel and the apparatus has an overall width of about 195 cm, a depth of about 186 cm (including the probe support), and an overall height of about 210 cm.

As mentioned, known cuboid and cylindrical support structures for 2000 L-3000 L vessels have heights that do not allow for installation in existing cleanrooms without interior modifications. Moreover, the height of, for example, a 2000 L/3000 L cubical/cuboid (e.g., square) tank cannot be sufficiently reduced may making it wider due to challenges (such as limitations in film widths) in manufacturing large cuboid flexible vessels for use within wider support structures. The use of a hexagonal flexible bioprocessing vessel construction, however, with a greater number of relatively small/narrow panels, allows for the creation of 2000 L-3000 L flexible bioprocessing vessels having a liquid level height to width ratio of less than or equal to approximately 1. This, in turn, facilitates the use of rigid support structures with heights (and widths) below those of existing standard cleanroom doors, e.g., a double door having a height of 213.4 cm (7 ft) and a width of 182.9 cm (6 ft).

While embodiments of the rigid body and flexible hexagonal bioprocessing vessel are not limited to a specific height or width, it is contemplated that the rigid body and hexagonal flexible bioprocessing vessel will have a capacity of 2000 L or greater while maintaining a height allowing passage through existing cleanroom doors as described above.

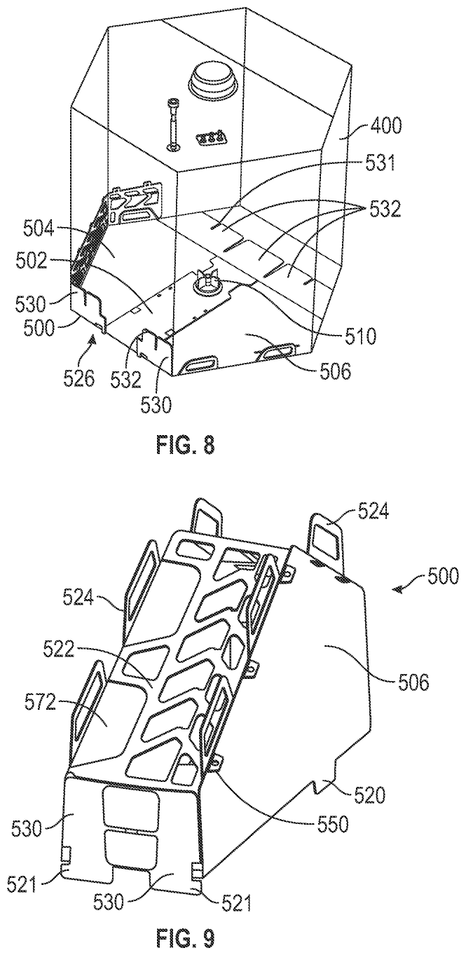

Referring now to FIG. 2, embodiments of the apparatus 100 facilitate alignment of the flexible bioprocessing vessel during installation. In particular, the bottom surface 106 of the rigid body 102 includes at least one alignment aperture 114, which in the depicted embodiment is two alignment apertures. The alignment apertures 114 are each shaped and configured to receive a tab 520 that is operatively attached to a bottom of the flexible vessel 400 (FIGS. 8 and 9). The alignment apertures 114 need not be any specific shape or size as long as they are capable of functionally engaging a feature on the vessel so that a user is alerted to proper alignment. The alignment apertures are generally equidistant from the drive motor head/impeller engagement mechanism 118. As will be appreciated, in embodiments, the alignment apertures 114 generally align the impeller of the vessel with the motor that engages the impeller to provide rotation, e.g., mixing.

The bottom surface 106 further includes openings 116 that allow for the passage of a drain line from a flexible bioprocessing vessel through the bottom surface 106 for connection to an external receptacle (not shown). In this regard, the rigid body 102 also includes a bracket 161 that is used to hold or support the drain line (FIG. 1). These features may take a variety of shapes and sizes provided a drain line can be effectively connected and supported for use.

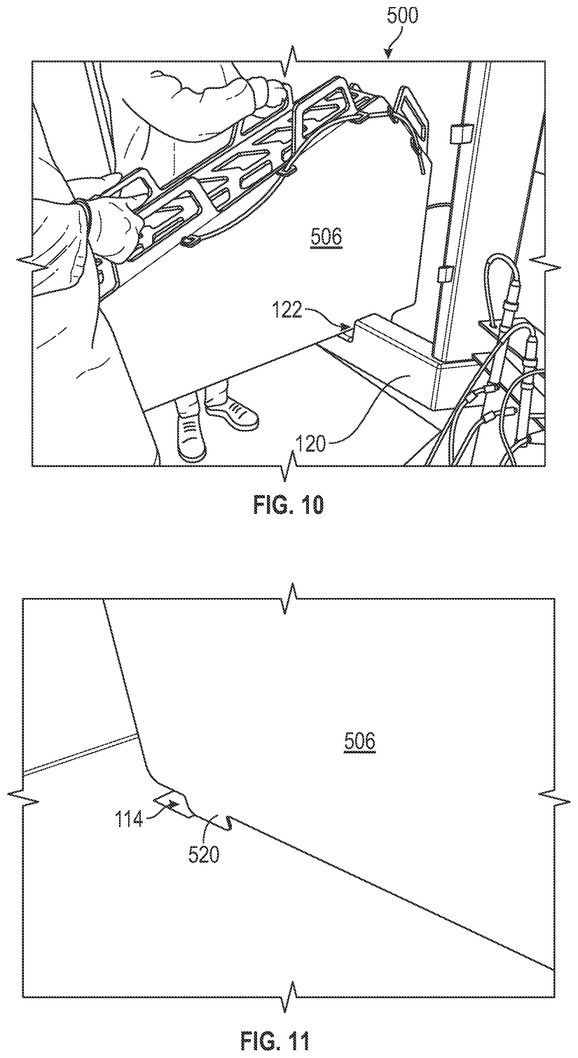

The bottom surface 106 further includes a slot 117 (or slots) that, in an embodiment, are open to or part of openings 116. The slots 117 are located behind a sill 120 which is shown in FIG. 5 and FIG. 10. The slots 117 are also not limited in size or shape provided they allow for sufficient locating/alignment functionality. As described in greater detail below, the alignment apertures 114 and slots 117 are used to align and install a vessel within the hexagonal interior 104.

In embodiments, the rigid body 102 may be equipped with heating and cooling which may be in the form of a jacket (not shown). To that end, the rigid body includes heating inlet 130 and a heating outlet 131. The rigid body 102 further includes one or more mounting brackets 132 to secure a controller and/or cabinet containing pumps, load cells connections, and the like to the body (not shown).

In an embodiment, the rigid body 102 is manufactured from stainless steel. The invention is not so limited, however, and other suitably rigid and durable materials may be employed. In certain embodiments, the rigid body 102 may be manufactured from a polymeric material. The rigid body 102 included panels that are welded together or joined by mechanical and/or chemical fastening mechanisms. In certain embodiments, the rigid body 102 may be cast or molded and may therefore be unitary.

While the six rigid panels 108 are depicted as being equal in size, in certain embodiments, the panels may vary in width. That is, for example, it may be possible for two opposing panels to be of a first width and the remaining panels a second width that is greater or less than the first width. Likewise, the six rigid panels 108 need not each be unitary and each panel could be composed of two or more joined/welded subpanels. The rigid panels are also not limited to a specific thickness and may be a multilayer composite or may be a single layer of rigid material. In embodiments, the rigid panels may also include insulative material.

In certain embodiments, the panels may be removably joined together so that the rigid body may be disassembled after use for storage and/or shipped as a flat package for assembly on site.

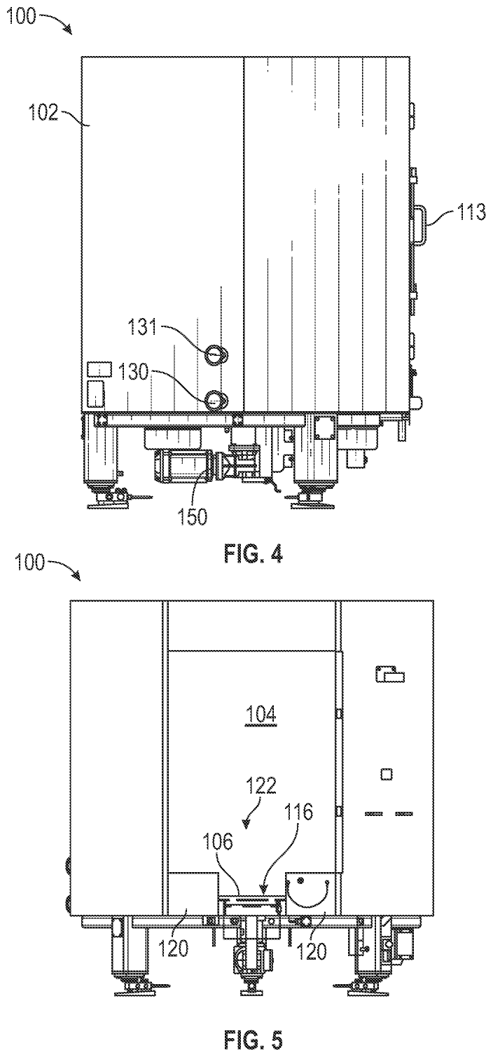

Referring now to FIG. 3 and FIG. 4, the rigid body 102 further includes a seat locking handle/mechanism 140 which is used to clamp the impeller seat on the vessel (not shown) to the rigid body 102. The door 112 further includes a latching mechanism and handle 113, which, in embodiments, may include a locking device or sensor to provide an alert if opened during use. The apparatus 100 is also depicted with a mixing motor 150 operatively connected to the rigid body 102.

Turning now to FIG. 5, which depicts a rigid body 102 with the selectively openable door removed, embodiments of the invention include, as previously mentioned, a sill 120. The sill 120 is located between the selectively openable door 112 and the hexagonal interior 104. The sill 120 has a rectangular void or notch 122 that is configured to receive the hexagonal flexible bioprocessing vessel in a folded state to facilitate installation of the vessel on the bottom surface 106 of the hexagonal interior 104. Thus, the notch 122 provides an initial alignment feature guiding the vessel into the hexagonal interior 104. In embodiments, the notch 122 is sized to approximate a width of a base portion 502 of a support apparatus that is used to transport and install the vessel (FIGS. 8 and 9). In one embodiment, the notch 122 has a width sized to accommodate passage of a base portion 502 having a width of 34 cm.

The sill 120 is not limited to a specific thickness, shape, or dimension. However, the notch 122 in the sill 120 should be configured to allow passage of the folded vessel while generally guiding it toward alignment features, e.g., alignment apertures 114 on the bottom surface 106 of the hexagonal interior 104.

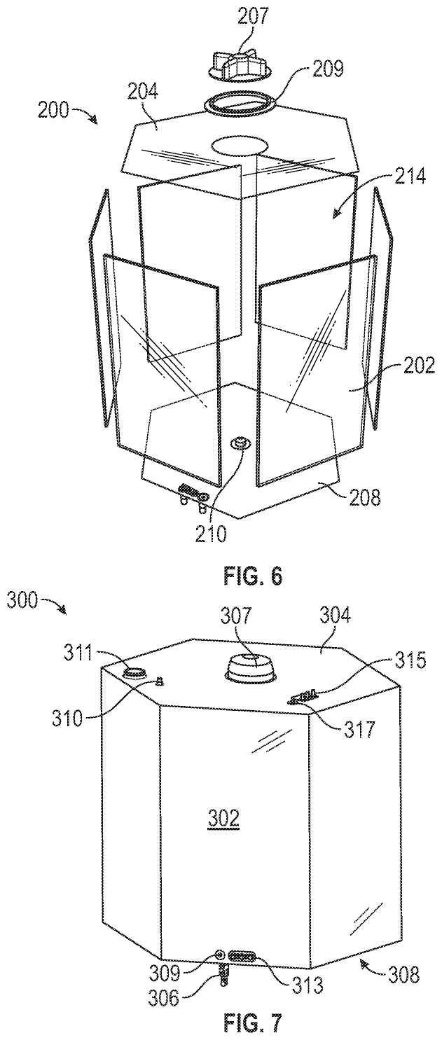

Embodiments of the invention are also directed to hexagonal bioprocessing vessels. As shown in FIG. 6, in one embodiment, a hexagonal flexible bioprocessing vessel 200 includes six flexible panels 202 that are secured to a top panel 204 (which, in embodiments, may be a two-panel design (FIG. 8)) and a bottom panel 206 that define an interior cavity 214 configured for processing a fluid.

The interior cavity 214 includes an impeller 210 (here the impeller seat, which is welded to the vessel, is shown without blades) and the top panel 204 includes a protective cap 207, which is attached to the vessel via a flange 209. The protective cap 207 covers and protects the impeller 210 during shipping/transport of the vessel.

As will be appreciated, embodiments are not limited to any specific impeller design or configuration. Assuming, for example, desired mixing power and minimum mixing/agitation volumes are attained, a variety of impellers may be utilized. An example of an impeller design suitable for use with embodiments of the invention is described in U.S. Pat. No. 11,065,589, which is incorporated by reference in its entirety, except to the extent that any of the incorporated material is inconsistent with the express disclosure herein.

In embodiments, the six flexible panels, top panel and bottom panel are each thermally welded to adjacent panels. As will be appreciated, a variety of manufacturing techniques and processes may be employed to assemble, weld, or otherwise construct the inventive flexible bioprocessing vessel and the embodiments are not limited in this regard. Although depicted as being constructed from nine separate panels that include separate bottom and top panels, it may be possible for the bottom and/or top to be integrated into the six flexible panels 302 that form the sides of the vessel.

Referring now to FIG. 7, an assembled hexagonal flexible bioprocessing vessel 300 configured for use with embodiments of the invention is depicted. The vessel 300 includes the aforementioned six flexible panels 302 which form the sides of the hexagonal flexible bioprocessing vessel. The flexible bioprocessing vessel 300 includes a single piece top panel 304 adjoining the six flexible panels 302 and forming a top of the hexagonal flexible bioprocessing vessel 300, and a bottom panel 308 located on an opposite end of the hexagonal flexible bioprocessing vessel 300 from the top panel 304, the bottom panel 308 adjoining the six flexible panels 302 and forming a bottom of the hexagonal flexible bioprocessing vessel 200. The six flexible panels 302, top panel 304, and bottom panel 308 define the interior cavity that holds fluid for mixing/processing.

The hexagonal flexible bioprocessing vessel 300 further includes at least one fluid input 310 and at least one fluid output (e.g., drain port 306) for adding and removing fluid to and from the interior cavity of the vessel respectively. In embodiments, the vessel 300 further includes a sample line port 309, a powder port 311, a one or more sensor ports 313, which may be mounted on a plate that is welded to the vessel. In certain embodiments, the vessel 300 includes inflation ports 315 used to inflate the bag during installation, and one or more dosing ports 317. The vessel also includes an impeller which is not shown (See e.g., FIG. 8).

Importantly, and as mentioned previously, the hexagonal geometry of the flexible vessel allows for the creation of 2000 L-3000 L flexible bioprocessing vessels having a liquid level (e.g., a maximum liquid level) height to width ratio of less than or equal to 1, which, in turn, allows for the construction and use of lower and wider rigid support structures/tanks.

In embodiments, a hexagonal flexible bioprocessing vessel has a 2000 L capacity and a liquid level height to width ratio of about 0.91. This 2000 L vessel has a width of about 150 cm, a height of about 151 cm, and a liquid level (e.g., maximum liquid level) height of about 137 cm. A 3000 L hexagonal flexible bioprocessing vessel according to an embodiment has a liquid level height to width ratio of about 0.86, a width of about 175 cm, an overall height of about 159 cm, and a liquid level height of about 151 cm.

In addition to facilitating the installation of rigid support structures in existing cleanroom/laboratory spaces, the hexagonal flexible vessel geometry has also been shown to facilitate efficient mixing with both floating and sinking powders providing a level of versatility not available with known cubical or cylindrical vessels and tanks.

Moreover, the hexagonal flexible bioprocessing vessels also have a large volume range. In a specific embodiment, a 2000 L hexagonal vessel has a nominal maximum volume of about 2000 L, a minimum agitation volume of about 24 L, a minimum sensing volume of about 51 L, and a minimum mixing volume of about 172 L. In another embodiment, a 3000 L hexagonal vessel has a nominal maximum volume of about 3000 L, a minimum agitation volume of about 36 L, a minimum sensing volume of about 62 L and a minimum mixing volume of about 236 L.

As will be appreciated, embodiments of the vessel may be manufactured from a variety of flexible materials, including various polymeric materials. The invention is not limited to any specific film material, thickness, or the like. The film may be a multi-layer composite or a single layer of material and may be opaque or transparent.

While the six flexible panels 202 are depicted as being equal in size, in certain embodiments, the panels may vary in size, e.g., width. That is, for example, two opposing panels may be of a first width and the remaining panels may be a second width greater or smaller than the first width. Likewise, the six flexible panels 202 need not be unitary and each panel could be composed of two or more joined/welded subpanels, similar to the top panel 204 shown in FIG. 6.

Referring now to FIG. 8, an embodiment of a 3000 L hexagonal flexible bioprocessing vessel 400 is depicted. As shown, the vessel is connected to an apparatus 500 for transporting and installing the vessel. The apparatus 500 is secured to the vessel via an external, bottom facing portion the impeller 510. The apparatus 500 includes a base portion 502 that includes a first wing portion 504 and a second wing portion 506, each being hingedly attached to the base portion 502. In use, the first wing portion 504 and the second wing portion 506 are selectively movable relative to the base portion 502 between a first position in which the apparatus is folded to form a storage cavity 572 for the vessel (FIG. 9) and a second position in which the apparatus is unfolded and substantially planar/flat within the support structure so that the vessel may be used.

In the first position, which is depicted in FIG. 9, the first wing portion 504 and the second wing portion 506 are angled relative to the base portion 502, e.g., at an approximate 90-degree angle, to form the storage cavity 572. The apparatus 500 further has handles 524 which facilitate transport and installation of the apparatus 500. In this position, the vessel is folded and located within the apparatus 500 and may be further packaged and shipped.

In an embodiment, at least one of the first wing portion 504 and the second wing portion 506 includes a side flap 522 that, in the first position, extends between the first wing portion 504 and the second wing portion 506 to define an upper surface of storage cavity 572. Each side flap 522 may include connector tabs 550 that fit through slots 552 in the opposite wing portion. The tabs 550 may include a hole configured to accept a zip tie or like connector to fix the wing portions and side flap 522 in place forming the storage cavity 572. In embodiments, each side flap 522 may include one or more integrated handles 524 that fold out when the apparatus 500 is in the first position. In any event, in embodiments, at least one of the first wing portion 504 and second wing portion 506 includes a handle 524.

Moreover, one of the base portion 502, the first wing portion 504, and/or the second wing portion 506 includes a front flap 530 and/or a rear flap 532, that, in the first position, define a front surface and/or a rear surface of the storage cavity, respectively. In the depicted embodiment, each of the first wing portion and second wing portion include a front flap 530. The front flaps may be joined together when the apparatus 500 is in the first position via slots 531.

As shown, in an embodiment, each of the front flaps 530 includes a tab or foot portion 521 which performs a locator/alignment function by dropping into slot(s) 117 behind the sill 120 of the rigid hexagonal support structure facilitating proper alignment of the vessel. The front flap(s) also include an open or cut away portion 526 that facilitates connection of one or more fluid lines and/or probes to the flexible bioprocessing vessel 400.

In the depicted embodiment, each of the first wing portion 504, second wing portion 506, and the base portion 502 also include a rear flap 532. These rear flaps 532 may be joined together with the rear flap 532 on the base portion 502 on the inside of the other two rear flaps via slots 531.

As will be appreciated, the first and second wing portions, base portion 502, and the side, front and rear flaps, form all sides of the apparatus 500 thereby enclosing the vessel.

Importantly, the first and/or second wing portion includes a locator tab 520. The locator tab 520 is configured to engage an alignment aperture 114 of the rigid body 102 to provide an indication to a user installing the hexagonal flexible bioprocessing vessel that it is properly aligned in the hexagonal interior of the rigid body. While the locator tab 520 is depicted as part of a separate installation apparatus 500, in certain embodiments, the vessel itself may include a locator tab or other feature that engages an alignment aperture of the rigid body. Indeed, the apparatus 500 may be unitary with or otherwise a part of the flexible hexagonal bioprocessing vessel rather than a separate component attached thereto.

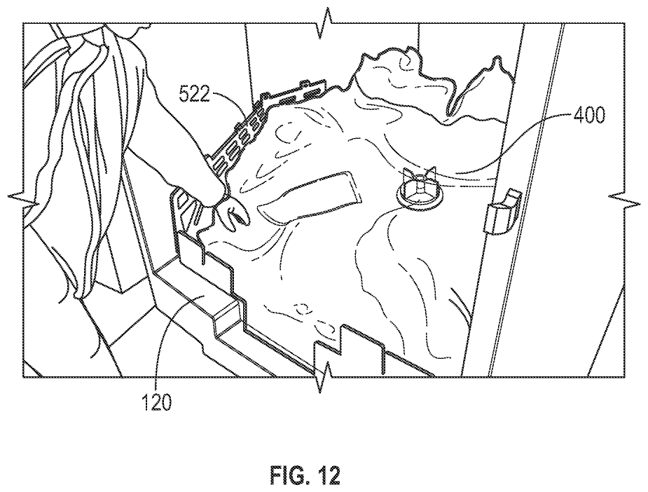

Referring now to FIG. 10, FIG. 11, and FIG. 12, an installation method is depicted in which two users will initially lift/free the apparatus 500 (which includes the vessel in the storage cavity) from external packaging (not shown). Generally, this will involve two users, one on each side of the base portion, gripping handles located on the wing portions. The apparatus 500 is then placed within the notch 122 in the sill 120 on the front edge of the hexagonal interior of the rigid body. The apparatus 500 fits into the notch 122 and is guided through into the hexagonal interior. The locator tabs 520 are then received by/engage the at least one alignment aperture 114, such that the apparatus 500 is in a substantially aligned position. Once this position is achieved, the seat locking mechanism 140 may be activated which engages the impeller seat (not shown) to lock the vessel in place.

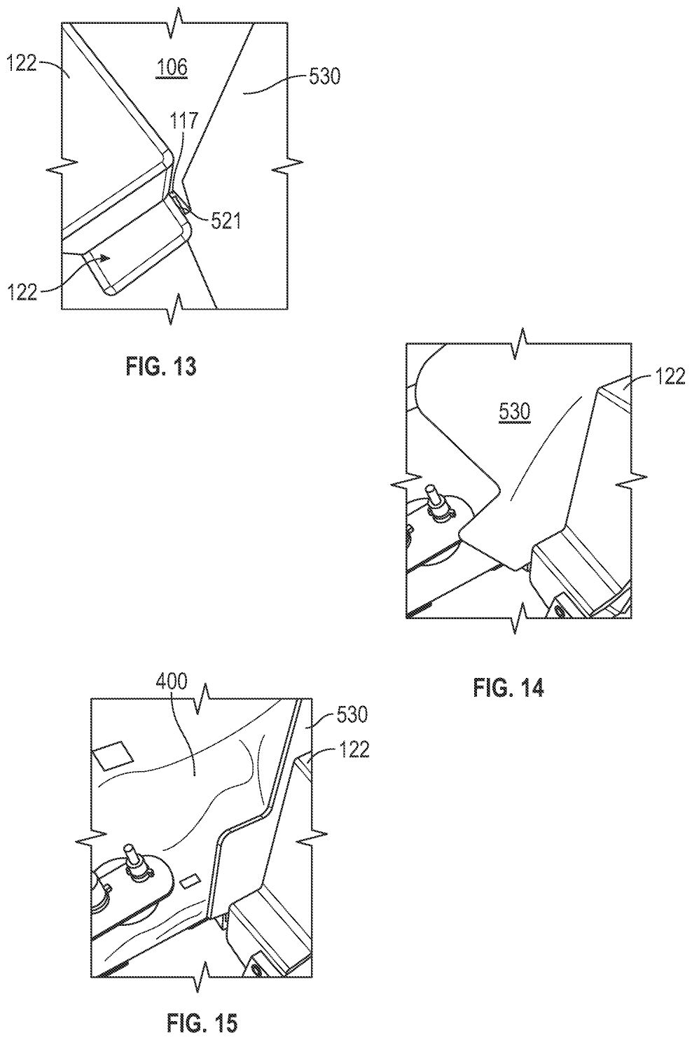

Referring now to FIG. 13, FIG. 14, and FIG. 15, once the apparatus 500 and vessel 400 are in the vessel and the tabs are in the alignment apertures (FIG. 11) and the seat lock activated, a user should check to ensure that each foot portion 521 of each front flap 530 is fully seated in one of the slots 117. If so, the front flaps 530 and wing portions may then be opened into the second position and installation may be completed by connecting all relevant fluid lines, pumps, and sensors and inflating the vessel. Use may then be commenced.

Embodiments of the invention also contemplate a method of folding a hexagonal flexible bioprocessing vessel for storage and transportation, such as within an apparatus 500 for transporting and installing the vessel. In a specific embodiment, the method involves placing the top panel flat on top of the bottom panel making sure that the impeller cover is positioned over the impeller to protect the vessel film. Then, the six corners of the top panel are pushed down onto the six corners of the bottom panel. At this stage, the six flexible panels that form the sides are substantially upright in a standing position.

Then, one by one, the panels on each side of a weld/seam between the six flexible panels are pushed together and each weld is folded over to an operator's left onto the top panel. This is repeated six times so the formerly upright sides are folded over onto the top panel. The resulting folded vessel now has a substantially hexagonal shape. At this point, the vessel may be folded again wherein opposing sides are folded together towards a center of the vessel with the fluid outlet (e.g., port) facing upward toward the operator. The vessel may now be placed into exterior packaging for shipment/transport.

As will be appreciated, despite the above, embodiments are not limited to a specific folding or packaging process or technique.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

1. An apparatus for supporting a flexible bioprocessing vessel comprising:
a rigid body having a hexagonal interior that includes a bottom surface and six sides surrounding the bottom surface, the hexagonal interior having a substantially open top;
a selectively openable door allowing access to the hexagonal interior; and
a stand portion attached to the rigid body, the stand portion allowing access to an underside of the rigid body;
wherein the hexagonal interior is configured to receive a hexagonal flexible bioprocessing vessel having a liquid level height to width ratio of 1.

2. The apparatus of claim 1 wherein the hexagonal interior is configured to receive a 2000 L hexagonal flexible bioprocessing vessel and the apparatus has an overall width of about 172 cm and an overall height of about 203 cm.

3. The apparatus of claim 1 wherein the hexagonal interior is configured to receive a 3000 L hexagonal flexible bioprocessing vessel and the apparatus has an overall width of about 195 cm and an overall height of about 210 cm.

4. The apparatus of claim 1 wherein the bottom surface has a least one alignment aperture configured to engage at least one locator tab on a base portion of the hexagonal flexible bioprocessing vessel to facilitate alignment of the hexagonal flexible bioprocessing vessel within the hexagonal interior.

5. The apparatus of claim 1 wherein rigid body has a hexagonal exterior formed by six rigid panels, the six rigid panels also defining the six sides of the hexagonal interior.

6. The apparatus of claim 5 wherein one of the six rigid panels faces a front of the apparatus and includes the selectively openable door.

7. The apparatus of claim 6 further comprising an opening in the rigid body located below the selectively openable door, the opening facilitating connection of one or more fluid lines and/or probes to the hexagonal flexible bioprocessing vessel.

8. The apparatus of claim 1 wherein the rigid body includes a sill located between the selectively openable door and the bottom surface of the hexagonal interior, the sill having a notch configured to receive the hexagonal flexible bioprocessing vessel in a folded or collapsed state to facilitate installation of the hexagonal flexible bioprocessing vessel on the bottom surface of the hexagonal interior.

9. The apparatus of claim 1, wherein the bottom surface includes at least one opening configured to allow tubing from the hexagonal flexible bioprocessing vessel to exit the hexagonal interior.

10. A hexagonal flexible bioprocessing vessel comprising:
six flexible panels forming sides of the hexagonal flexible bioprocessing vessel;
a top panel adjoining the six flexible panels, the top panel forming a top of the hexagonal flexible bioprocessing vessel;
a bottom panel adjoining the six flexible panels, the bottom panel located on an opposite end of the hexagonal flexible bioprocessing vessel from the top panel, and forming a bottom of the hexagonal flexible bioprocessing vessel, the six flexible panels, top surface, and bottom panel defining an interior cavity configured for processing a fluid; and
at least one fluid input and at least one fluid output for adding and removing fluid to and from the interior cavity of the hexagonal flexible bioprocessing vessel respectively;
wherein hexagonal flexible bioprocessing vessel has a liquid level height to width ratio of 1.

11. The hexagonal flexible bioprocessing vessel of claim 10 wherein the hexagonal flexible bioprocessing vessel has a 2000 L capacity and a liquid level height to width ratio of about 0.91.

12. The hexagonal flexible bioprocessing vessel of claim 11 wherein the hexagonal flexible bioprocessing vessel has a width of about 150 cm, an overall height of about 151 cm, and a liquid level height of about 137 cm.

13. The hexagonal flexible bioprocessing vessel of claim 10 wherein the hexagonal flexible bioprocessing vessel has a 3000 L capacity and a liquid level height to width ratio of about 0.86.

14. The hexagonal flexible bioprocessing vessel of claim 13 wherein the hexagonal flexible bioprocessing vessel has a width of about 175 cm, an overall height of about 159 cm, and a liquid level height of about 151 cm.

15. The hexagonal flexible bioprocessing vessel of claim 10 wherein the bottom panel and/or the top panel is a separate panel.

16. The hexagonal flexible bioprocessing vessel of claim 10 wherein each of the six flexible panels are thermally welded to adjacent panels.

17. The hexagonal flexible bioprocessing vessel of claim 10 wherein the interior cavity includes an impeller and the top panel includes a selectively removable impeller cover to protect the impeller during transportation of the hexagonal flexible bioprocessing vessel.

18. The hexagonal flexible bioprocessing vessel of claim 10 wherein the hexagonal flexible bioprocessing vessel may be folded for storage and/or transportation and unfolded for installation on a bottom surface of a rigid support structure.
